(19) United States
(12) Patent Application Publication
(10) Pub. No.: US 2026/0283456 A1
(43) Pub. Date: Sep. 24, 2026

(54) LARYNGOSCOPE WITH IMPROVED VISUALIZATION

(71) Applicant: The Regents of the University of Michigan, Ann Arbor, MI (US)

(72) Inventors: Matthew Frank Grzywinski, Ann Arbor, MI (US); Richard Moschella, Ann Arbor, MI (US); Maxwell D. Raithel, Ann Arbor, MI (US); Madison E. Daminato, Birmingham, MI (US); Alex S. Li, Lake Forest, IL (US); Elizabeth M. Mittag, Shelby Township, MI (US); Raj K. Patel, Redford, MI (US); Rithika P. Reddi, Ann Arbor, MI (US); Kathryn J. Suarez, Ann Arbor, MI (US)

(21) Appl. No.: 19/477,475

(22) PCT Filed: Apr. 22, 2024
(86) PCT No.: PCT/US2024/025707

§ 371 Date: Oct. 21, 2025

Related U.S. Application Data
(60) Provisional application No. 63/460,956, filed on Apr. 21, 2023.

(51) Int. Cl. A61B 1/12; A61B 1/00; A61B 1/015; A61B 1/05; A61B 1/267

(52) U.S. Cl. CPC A61B 1/126; A61B 1/00042; A61B 1/00066; A61B 1/00094; A61B 1/00096; A61B 1/00119; A61B 1/00128; A61B 1/015; A61B 1/05; A61B 1/267

(57) ABSTRACT

A laryngoscope includes a lens cleaner and/or a suction tube. The lens cleaner is configured to discharge a cleaning fluid onto a camera lens of the laryngoscope upon actuation by a user. The suction tube can be extended from and retracted toward a blade of the laryngoscope by the user. This construction permits the user to maintain visualization of a patient's airway when unexpected fluids or debris are encountered without removing the laryngoscope or resorting to an alternative intubation strategy. The lens cleaner and suction tube feeder can be actuated with the same hand of the user holding the laryngoscope.

### TECHNICAL FIELD

The present disclosure relates laryngoscopes and, in particular, to camera-equipped laryngoscopes and their use in performing intubation procedures.

### BACKGROUND

Intubation is the insertion of an endotracheal (ET) tube into a patient's trachea or airway to provide oxygenation and ventilation and is relatively common in hospital emergency departments, where it was performed over 346,000 times in 2020. Intubation involves sedating and inducing paralysis in the patient, followed by use of a laryngoscope to visualize the entrance to the patient's airway for proper placement of the ET tube. The laryngoscope includes a tongue blade affixed to a handle which the operator uses to insert and manipulate the blade in the patient's mouth. The tongue blade repositions the tongue and widens the space for the ET tube, which is subsequently passed into the airway. In direct laryngoscopy (DL), the user establishes a direct line-of-sight with their eyes to visualize the airway entrance. Laryngoscopy blades used in DL are typically straight with an angled tip or slightly curved, as with standard Miller or MacIntosh (Mac) blade geometries. In contrast, video laryngoscopy (VL) uses a camera to indirectly visualize the airway entrance via a video monitor or screen, for example. Laryngoscopy blades in used in VL can be one of the standard geometries or a hyperangulated blade with a greater amount of curvature than a Mac blade. Hyperangulated blades practically require indirect visualization due to their geometry.

Video laryngoscopy with a hyperangulated blade can offer better visualization of airway structures in patients with difficult body habitus such as obesity, head or neck trauma, limited neck mobility, or abnormal airway anatomy. A camera on the laryngoscope permits vocal structures not in the operator's line of sight to be viewed and decreases procedure complications without increasing procedure time. Coupled with the additional curvature of the hyperangulated blade, the operator can often visualize airway structures that would be challenging or even impossible to see with Mac or Miller blades.

One limitation of video laryngoscopy is potential fouling of the camera lens in a soiled airway, which is characterized by the presence of blood, vomitus, or other secretions at the airway entrance. Fouling of the camera lens defeats the visualization function and requires withdrawal of the blade for cleaning or a transition to direct laryngoscopy. It is estimated that nearly one-third of ET intubations in hospital emergency departments are performed in soiled airways. Soiled airways reduce first pass success with VL, and multiple attempts require more time than a single attempt in a procedure where time is of the essence. Unfortunately, it is not possible to reliably predict the extent of airway contamination prior to laryngoscopy, or whether a clear airway will become soiled during the procedure.

### SUMMARY

Embodiments of a laryngoscope include a lens cleaner.

In various embodiments, the laryngoscope includes a camera lens along an exterior surface of the laryngoscope, and the lens cleaner directs a stream of cleaning fluid onto the camera lens when the lens cleaner is actuated.

In various embodiments, the lens cleaner includes a discharge port and a conduit configured to place a cleaning fluid source in fluidic communication with the discharge port such that cleaning fluid is discharged toward a lens of the laryngoscope when the cleaning fluid source is pressurized.

In various embodiments, the laryngoscope includes a housing that houses the lens cleaner. The housing is configured to receive a separate cleaning fluid source to form a fluid-tight connection between the cleaning fluid source and a conduit of the laryngoscope such that cleaning fluid is discharged from the laryngoscope when the cleaning fluid source is pressurized.

In various embodiments, the cleaning fluid source is a syringe.

In various embodiments, the lens cleaner discharges a liquid cleaning fluid.

In various embodiments, the laryngoscope includes a handle, a blade extending radially from the handle, and a camera lens mounted along the blade. The lens cleaner includes a discharge port located along the blade between the camera lens and a distal end of the blade and offset from an axis of the camera lens to provide cleaning fluid on the camera lens when the lens cleaner is actuated.

In various embodiments, the blade is hyperangulated.

In various embodiments, the laryngoscope includes a suction tube arranged to remove debris and/or excess liquid from an airway of a patient during use.

In various embodiments, a position of an end of the suction tube with respect to a blade of the laryngoscope is adjustable to permit a user to control placement of the distal end of the suction tube relative to an airway of a patient during use.

In various embodiments, the laryngoscope includes a handle, by which a user manipulates a position and orientation of the blade relative to the airway of the patient while holding the handle with a hand, and a camera lens mounted along the blade. An actuator of the lens cleaner and an actuator that controls placement of the suction tube are operable by the same hand holding the handle.

Embodiments of a laryngoscope include an extendable and retractable suction tube.

In various embodiments, the laryngoscope includes a lens cleaner.

In various embodiments, the laryngoscope includes an actuator configured to selectively extend and retract the suction tube with respect to a blade of the laryngoscope.

In various embodiments, the actuator is operable to feed the suction tube along a housing of the laryngoscope and through an opening along the blade.

In various embodiments, the actuator is operable by a hand of a user holding a handle portion of the housing, by which the user manipulates a position and orientation of the blade relative to an airway of a patient during use.

In various embodiments, the suction tube is extendable into and retractable out of a field of view of a camera of the laryngoscope.

Embodiments of an intubation method include manually positioning a camera of a hyperangulated laryngoscope to visualize an airway of a patient, and cleaning a lens of the laryngoscope while the laryngoscope is positioned to visualize the airway of the patient.

In various embodiments, the method includes the step of extending a suction tube from a blade of the laryngoscope to remove debris and/or excess liquid from the airway.

In various embodiments, the step of cleaning and the step of extending are performed using the same hand used in the step of manually positioning.

### BRIEF DESCRIPTION OF THE DRAWINGS

FIG. 1 is a perspective view of a blade side of an illustrative laryngoscope;

FIG. 2 is a perspective view of a handle side of the laryngoscope of FIG. 1;

FIG. 3 is a side cutaway view of the laryngoscope of FIGS. 1 and 2;

FIG. 4 is a perspective view of an end of a blade of the laryngoscope of FIGS. 1-3, illustrating the relative positions and orientations of a camera, lens cleaner, and suction tube along the blade;

FIG. 5 is a photographic image of a working embodiment of the laryngoscope during actuation of the lens cleaner; and

FIG. 6 is a photographic image is the laryngoscope of FIG. 5 during actuation of the adjustable suction tube.

### DESCRIPTION OF EMBODIMENTS

Described below is a camera-equipped laryngoscope that includes lens cleaning functionality paired with an adjustable suction tube to allow a user to clear a soiled airway and clean a fouled camera lens without removing the laryngoscope from the patient's mouth. While these novel features can be used together to increase first-pass intubation success rates in soiled airways, it is contemplated that each feature can be individually incorporated into a laryngoscope to achieve its own improved function over conventional laryngoscopes. The laryngoscope enables medical professionals to perform video-assisted intubations with confidence that the procedure will not fail due to a fouled camera lens or an unpredictably soiled airway and reduces or eliminates the distraction of unexpected quick decision-making regarding a change in intubation strategy when a soiled airway is encountered.

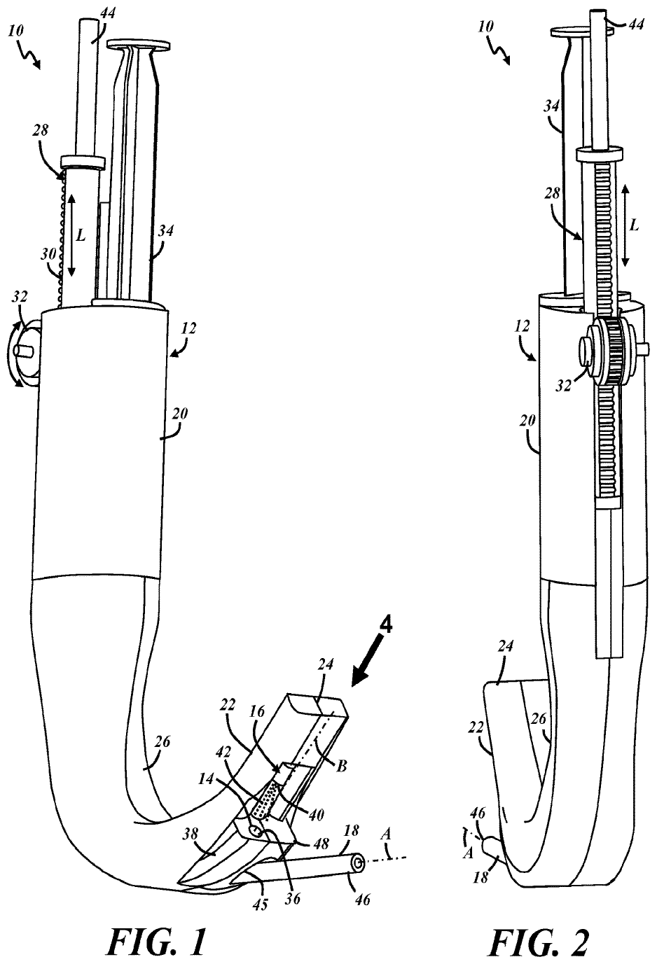

FIGS. 1 and 2 are respective views of a blade side and an opposite handle side of an embodiment of a laryngoscope 10. The laryngoscope 10 includes a housing 12, a camera 14, a lens cleaner 16, and a suction tube 18. The housing 12 includes a handle portion 20 and a blade portion 22 extending from the handle portion. The handle 20 is sized and shaped for manual gripping about its perimeter by a user, and the blade 22 is sized and shaped in the conventional manner of a laryngoscope blade. The illustrated blade 22 is a hyperangulated blade, which is generally characterized by a smaller radius of curvature between the handle 20 and an end 24 of the blade, as compared to a Macintosh blade, for example. The end 24 of the hyperangulated blade may also form a smaller angle with a longitudinal axis of the handle 20 than a Miller or MacIntosh blade does.

In this example, the blade 22 is a continuous extension of the handle 20 such that there is no defined point where the handle ends and where the blade begins. Rather, there is a gradual transition region 26 leading from handle 20 to the blade 22. The housing 12 may be formed by two opposing halves attached together as shown, each with a respective handle portion and blade portion made from a single monolithic piece, such as injection molded or 3D-printed plastic. Other constructions are possible. In addition to its handle and blade functions, the housing 12 may also house, support, or be configured to receive other components or portions of components of the laryngoscope 10. The housing 12 may define an internal hollow space or cavity to serve some of those functions and may also define one or more through-openings connecting the internal hollow space with an exterior surface of the housing. The illustrated housing 12 supports and partly houses the camera 14 and its associated cables, supports and partly houses the lens cleaner 16, partly houses the suction tube 18, and supports and partly houses a tube feeder 28 that includes a sleeve 30 and an actuator 32. The housing 12 is also configured to receive, support, and partly house a cleaning fluid source 34, which in this case is a syringe.

A lens 36 of the camera 14 is supported by the housing 12 along an exterior surface of the laryngoscope 10. In particular, the camera 14 is mounted with its lens 36 at an aperture formed through the housing 12 along a convex side of the blade 22, which is a side of the blade facing away from the handle 20. The camera 14 is mounted in a sub-housing 38 protruding from the convex side of the blade 22 with an axis of the lens 36 generally parallel with the end 24 of the blade.

The lens cleaner 16 includes a cleaning fluid discharge port 40 configured to direct a stream of cleaning fluid 42 onto the camera lens 36 when the lens cleaner is actuated. In this case, actuation is performed by depressing a plunger of the syringe 34 containing cleaning fluid, which pressurizes the cleaning fluid source. The discharge port 40 is located between the camera 14 and the end 24 of the blade 22 and faces the camera lens 36. The portion of the lens cleaner 16 defining the discharge port 40 is offset from the axis of the camera lens 36 so as not to obstruct the camera's field of view. Other actuation techniques are contemplated, such as opening a valve to release pre-pressurized fluid from a fluid source or electronic actuation of an electric pump. The cleaning fluid 42 is water, saline, or another biocompatible liquid. The cleaning fluid may also be a gas such as air, nitrogen, or an inert gas. More than one cleaning fluid may be employed, such as a liquid cleaning fluid to clear the camera lens 36 and a gas to clean residual liquid cleaning fluid from the lens.

The suction tube 18 extends from a first end 44 protruding from the handle portion 20 of the housing 12, through the hollow interior of the housing, to a second or distal end 46 able to protrude from the blade portion 22 of the housing. The first end 44 is adapted for attachment to a vacuum (negative pressure) source. The distal end 46 may be formed from a material that is more rigid than the remainder of the suction tube 18. The distal end 46 may be provided by a semi-rigid plastic tube or nozzle, for example, while the remainder of the suction tube 18 may be formed from a more flexible plastic or elastomer, such as polyurethane or silicone.

The suction tube 18 extends from the blade 22 of the housing 12 alongside and laterally offset from the camera 14. In particular, the suction tube 18 extends through an opening 45 in the housing 12 along the convex side of the blade 22. The opening 45 in the housing 12 is formed in a sub-housing 48 protruding from the convex side of the blade 22 and adjacent sub-housing 38 with an axis of extension A diverging from the end 26 of the blade and a camera axis B.

The suction tube 18 is extendable and retractable with respect to the housing 12. In this example, the suction tube 18 is moveable back and forth within the housing 12 such that the distance between the blade 22 and the distal end 46 of the suction tube is variable and controllable by the user by operating the tube feeder 28. The sleeve 30 of the tube feeder 28 fits snugly about the perimeter of the suction tube 18 such that the suction tube 18 moves together with or is stationary with respect to the surrounding sleeve 30. In this example, the sleeve 30 is slidingly engaged with the housing 12 for translational movement with respect to the housing in a longitudinal direction L of the handle 20. The actuator 32 in this embodiment is a thumb wheel that engages teeth formed along an exterior of the sleeve 30 in the manner of rack-and-pinion gears such that rotational motion of the actuator 32 results in tangential translation of the sleeve 30.

While a suction tube may be conventionally used to clear the airway of a patient being intubated, this often entails insertion by the user of a separate suction tube into the patient's mouth alongside the laryngoscope blade using the hand not holding the handle of the laryngoscope. The suction tube must then be manually withdrawn or held in place by some other means so that the user's spare hand is free to place the endotracheal tube in the airway. The ability to extend and retract the suction tube 18 with the same hand already holding the handle 20 of the laryngoscope eliminates the need to change items in the user's spare hand or otherwise take attention away from the intubation task. Additionally, extension of the suction tube 18 from the blade 22 alongside the camera 14 is particularly advantageous because the end 46 of the tube conveniently enters the field of view of the camera 14 as it is extended from the blade and then leaves the field of view of the camera in the opposite direction as it is retracted. The user is thus always aware of the current location of the distal end 46 of the suction tube 18. The distal end 46 of the suction tube 18 can be fully retracted into the housing 12 of the laryngoscope 10.

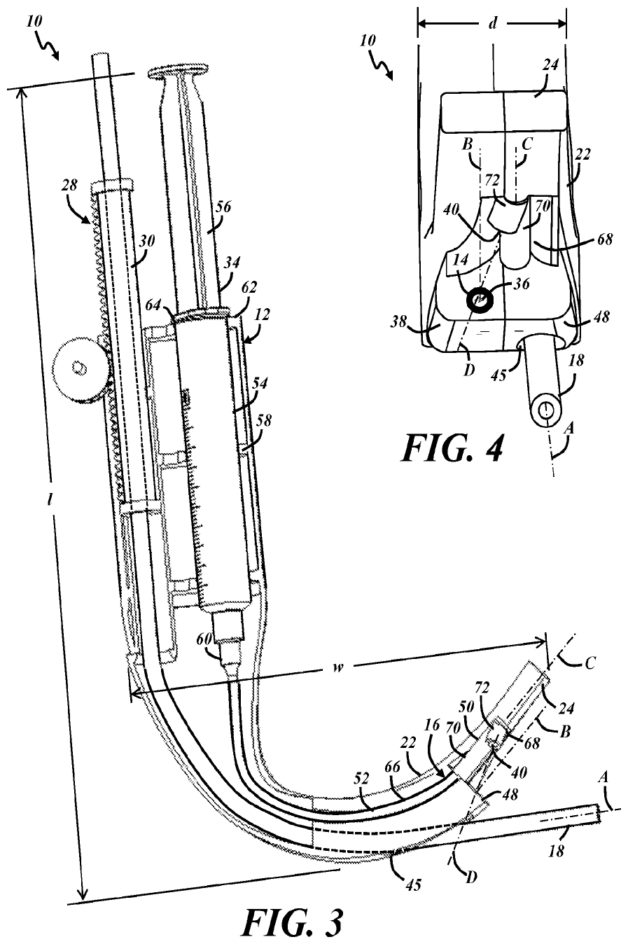

FIG. 3 is a cutaway view along a longitudinal midline of the laryngoscope 10 of FIGS. 1 and 2 illustrating additional features and showing already-described features from a different perspective. Here, the lens cleaner 16, suction tube 18, and tube feeder 28 are more fully illustrated. The inner surface of the housing 12 includes ribs 58 or other locating features shaped to secure the fluid source 34 within the housing via contact with a perimeter of the fluid source. Similar ribs or features along the inside of the housing 12 are provided as guide surfaces used in the sliding movement of the feeder sleeve 30 within the housing 12.

The lens cleaner 16 includes the cleaning fluid source 34 and a nozzle or sprayer 50 coupled with the fluid source via a cleaning fluid conduit 52. In this case, the cleaning fluid source 34 is a syringe including a barrel 54 and a plunger 56. This implementation of the cleaning fluid source 34 is practical in that it uses a commercially available syringe (e.g., 10 mL.) commonly available as a hospital or other medical office supply item. The fluid source 34 may be any feature with an internal volume capable of containing cleaning fluid and delivering the cleaning fluid to the discharge port 40 when its contents are pressurized or otherwise forced to flow.

In one embodiment, the cleaning fluid source 34 is omitted and the housing 12 is configured to receive a separately provided fluid source prior to use. For example, a luer lock fitting 60 coupled with a receiving end of the cleaning fluid conduit 52 may be affixed along an inner wall of the housing 12 at a predetermined distance from a receiving end 62 of the housing corresponding to a barrel length of a standard syringe 34. A technician can then insert the barrel of a pre-filled syringe through an opening 64 at the receiving end 62 of the housing 12 and twist the syringe to form a fluid-tight connection with the lens cleaner 16. In other examples, the cleaning fluid source 34 is an external source, and the laryngoscope 10 includes a connector configured to couple the external fluid source with the lens cleaner 16.

The cleaning fluid conduit 52 is a segment of flexible tubing in this example with its receiving end coupled with the fluid source 34 via a luer lock or other fluid-tight connection and its opposite end coupled with the nozzle 50. At least a portion of the fluid conduit 52 may extend along an internal channel 66 of the blade 22 of the housing 12 as shown in FIG. 3. The end of the conduit 52 coupled with the nozzle 50 is located at a recess 68 in the convex side of the blade 22, and the recess houses the nozzle. The recess 68 has a depth sufficient to support the nozzle 50 such that the nozzle is out of the field of view of the camera 14. The conduit 52 can take other or additional forms, such as a channel integrally formed in the blade 22 or housing 12.

The sprayer 50 includes the cleaning fluid discharge port 40 and, in this example, is an elbow conduit including first and second portions 70, 72. The first portion 70 is a straight tubular portion coupled with the conduit 52 at one end and coupled with the second portion 72 of the sprayer 50 at the other end. A longitudinal axis C of the first portion 70 of the sprayer 50 is generally parallel with the end 24 of the blade 22 and with the axis B of the camera 14. The second portion 72 of the sprayer 50 extends from the first portion 70 and includes the discharge port 40 at its opposite end. An axis D of the second portion 72 of the nozzle 50 forms an obtuse angle with the axis C of the first portion 70 and intersects the lens of the camera 14. The second portion 72 of the sprayer 50 extends from the first portion 70 at an acute angle such that, when pressurized, the cleaning fluid flows in a first direction along axis C through the first portion 70 and then in a second direction along axis D through the second portion 72, with the second direction having a component opposite to that of the first direction. In other words, the fluid is discharged from the sprayer 50 in a direction generally opposite that in which it enters the sprayer. In the illustrated example, the acute angle between axes C and D is in a range from 50 to 60 degrees.

FIG. 4 is a perspective view of the end 24 of the blade 22 from vantage point 4 of FIG. 1. This view illustrates several of the previously described features, including the suction tube 18 extending from an opening 45 of sub-housing 48 along the blade 22, the lens 36 of the camera 14 positioned in an aperture of sub-housing 38 along the blade, and the first and second portions 70, 72 of the sprayer of the lens cleaner 16 housed in recess 68 along the blade with its discharge port 40 directed toward the camera lens 36.

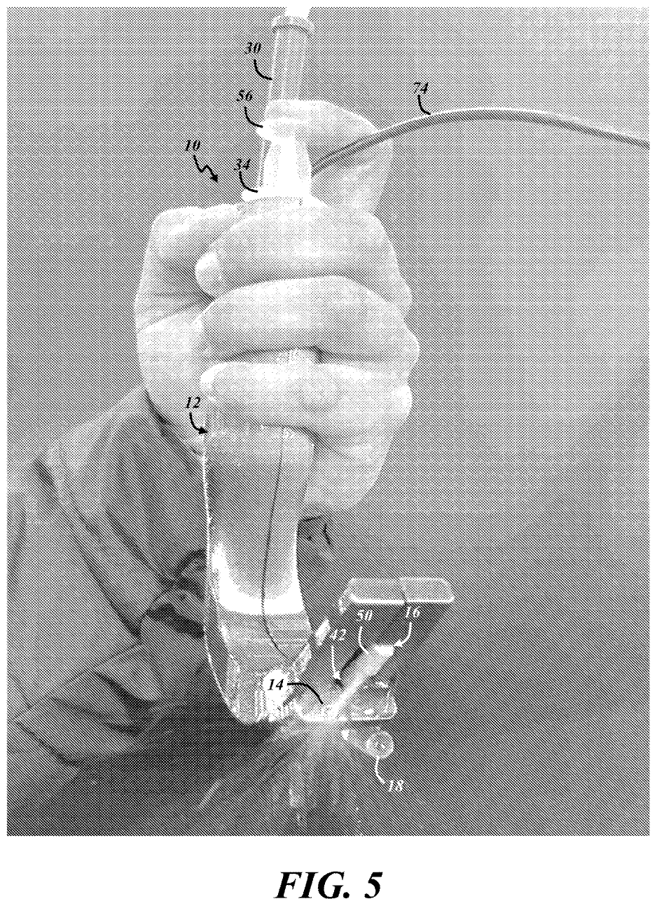

FIG. 5 is a photographic image of a working prototype of the above-described laryngoscope 10 during actuation of the lens cleaner 16. Here, a user is applying a force to the plunger actuator 56 of the syringe fluid source 34 to pressurize the cleaning fluid in the syringe, which results in a stream of cleaning fluid 42 being discharged from the sprayer 50 and onto the lens of the camera 14. Also visible in this image are the distal end of the suction tube 18 partially extended from the blade 22, the sleeve 30 of the suction tube feeder, and a cable 74 that extends from the camera 14 and through the housing 12 to connect to a video monitor and/or camera power source. FIG. 5 further illustrates another advantageous feature of the laryngoscope 10, which is that the lens cleaner 16 can be actuated with the same hand holding the handle 20 of the device.

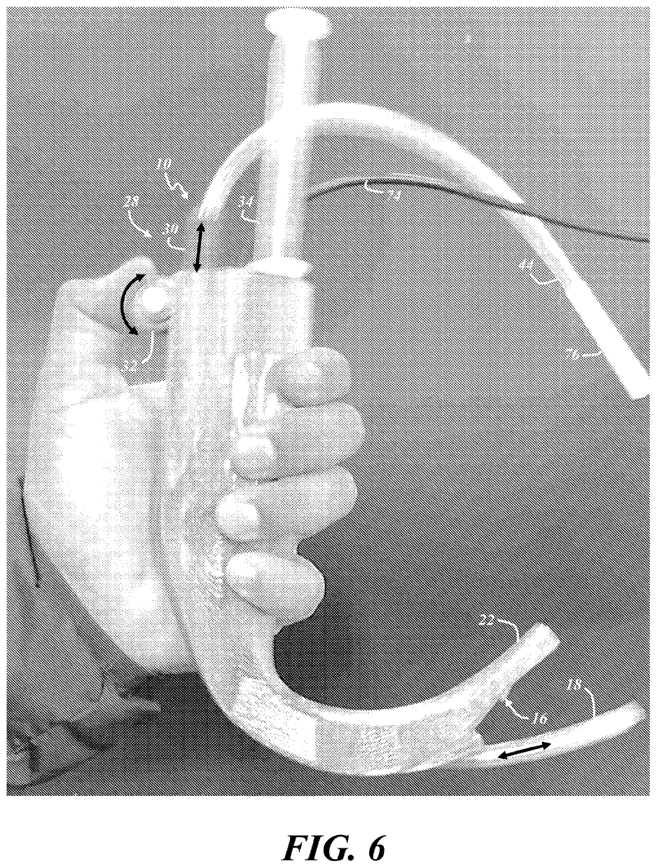

FIG. 6 is another photographic image of the prototype of FIG. 5 during actuation of the tube feeder 28. Here, the user is applying a torque to the thumbwheel actuator 32 of the tube feeder 28 to move the sleeve 30 back and forth along the housing 12, thereby extending or retracting the suction tube 18 away from or toward the blade 22. Also visible in this image are the camera cable 74 and a connector 76 at the first end 44 of the suction tube 18 adapted to couple the suction tube with a vacuum source. FIG. 6 further illustrates another advantageous feature of the laryngoscope 10, which is that the tube feeder 28 can be actuated with the same hand holding the handle 20 of the device and the same hand that actuates the lens cleaner 16 when necessary.

To provide an idea of the size scale of the device, non-limiting dimensions of the laryngoscope 10 may include a length/≤255 mm, a width w≤125 mm, and a depth d≤100 mm (see FIGS. 3 and 4). The prototype device of FIGS. 5 and 6 has a length/=255 mm, a width w=129 mm, and a depth d=24 mm. A useful design guideline is locating the manual contact point of each of the actuators (e.g., thumbwheel 32 and plunger 56) within 3 inches or less of the intended hand location along the handle 20. These dimensions are merely illustrative and may vary significantly depending on the size of the intended patient (e.g., pediatric, average adult, large adult, etc.).

The above-described laryngoscope 10 is useful in performing an intubation method that includes manually positioning the camera 14 of the laryngoscope to visualize the airway of a patient, and cleaning the lens 36 of the camera while the laryngoscope is positioned to visualize the airway of the patient. In other words, there is no need to remove the laryngoscope from the patient's mouth in order to clean the camera lens. The method may also include extending the suction tube 18 from the blade 22 of the laryngoscope 10 to remove debris and/or excess liquid from the airway. Advantageously, cleaning the lens and extending the suction tube to remove debris from the airway can be performed with the hand that is already holding the laryngoscope.

A laryngoscopy system may include the above-described laryngoscope 10 with any technically feasible combination of disclosed features, along with a video monitor to receive and display imagery obtained from the camera 14, and a vacuum source coupled with a fluid receptacle. The proximal or first end 44 of the suction tube is couple with vacuum source and receptacle. The vacuum source and fluid receptacle may be of the type commonly found in hospital emergency departments or operating rooms.

The above-described laryngoscope 10 may include other features not explicitly illustrated in the figures. For instance, the laryngoscope may be built as a single-use hyperangulated laryngoscope that, after use, can be unplugged from the video monitor and from the vacuum source and disposed of. The relatively small list of low-cost components required to make the laryngoscope enables its one-time use capability. The laryngoscope 10 may also include a light source positioned along the blade 22 with the emitted light directed in the same direction as the camera 14. In some embodiments, the camera 14 includes an integrated light source. Additionally, the lens cleaning and suction tube adjustability functions can be separately or distinctly implemented as part of the laryngoscope. In one example, the laryngoscope 10 is equipped with a lens cleaner 16 with a static suction tube 18 having a distal end at a fixed position relative to the blade 22, or the laryngoscope is equipped with a suction port opening on a surface of the blade rather than a suction tube. Alternatively, a laryngoscope used in line-of-sight, direct laryngoscopy (DL) may be equipped with the above-described adjustability function of the suction tube, which has its own distinct advantages due to the angle at while the tube is advanced from the blade 22. Further, there may be other combinations of features disclosed herein that offer advantages even without the lens cleaner or adjustable suction tube.

It is to be understood that the foregoing description is of one or more embodiments of the invention. The invention is not limited to the particular embodiment(s) disclosed herein, but rather is defined solely by the claims below. Furthermore, the statements contained in the foregoing description relate to the disclosed embodiment(s) and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Further, the term “electrically connected” and the variations thereof is intended to encompass both wireless electrical connections and electrical connections made via one or more wires, cables, or conductors (wired connections). Other terms are to be construed using their broadest reasonable meaning unless they are used in a context that requires a different interpretation.

1. A laryngoscope comprising a lens cleaner.

2. The laryngoscope of claim 1, further comprising a camera lens along an exterior surface of the laryngoscope, wherein the lens cleaner directs a stream of cleaning fluid onto the camera lens when the lens cleaner is actuated.

3. The laryngoscope of claim 1, wherein the lens cleaner comprises a discharge port and a conduit configured to place a cleaning fluid source in fluidic communication with the discharge port such that cleaning fluid is discharged toward a lens of the laryngoscope when the cleaning fluid source is pressurized.

4. The laryngoscope of claim 1, further comprising a housing that houses the lens cleaner, the housing being configured to receive a separate cleaning fluid source to form a fluid-tight connection between the cleaning fluid source and a conduit of the laryngoscope such that cleaning fluid is discharged from the laryngoscope when the cleaning fluid source is pressurized.

5. The laryngoscope of claim 4, wherein the cleaning fluid source is a syringe.

6. The laryngoscope of claim 1, wherein the lens cleaner discharges a liquid cleaning fluid.

7. The laryngoscope of claim 1, further comprising:
a handle;
a blade extending radially from the handle; and
a camera lens mounted along the blade,
wherein the lens cleaner comprises a discharge port located along the blade between the camera lens and a distal end of the blade and offset from an axis of the camera lens to provide cleaning fluid on the camera lens when the lens cleaner is actuated.

8. The laryngoscope of claim 7, wherein the blade is hyperangulated.

9. The laryngoscope of claim 1, further comprising a suction tube arranged to remove debris and/or excess liquid from an airway of a patient during use.

10. The laryngoscope of claim 9, wherein a position of an end of the suction tube with respect to a blade of the laryngoscope is adjustable to permit a user to control placement of the distal end of the suction tube relative to an airway of a patient during use.

11. The laryngoscope of claim 10, further comprising:
a handle by which a user manipulates a position and orientation of the blade relative to the airway of the patient while holding the handle with a hand; and
a camera lens mounted along the blade,
wherein an actuator of the lens cleaner and an actuator that controls placement of the suction tube are operable by the same hand holding the handle.

12. A laryngoscope comprising an extendable and retractable suction tube.

13. The laryngoscope of claim 12, further comprising a lens cleaner.

14. The laryngoscope of claim 12, further comprising an actuator configured to selectively extend and retract the suction tube with respect to a blade of the laryngoscope.

15. The laryngoscope of claim 14, wherein the actuator is operable to feed the suction tube along a housing of the laryngoscope and through an opening along the blade.

16. The laryngoscope of claim 15, wherein the actuator is operable by a hand of a user holding a handle portion of the housing, by which the user manipulates a position and orientation of the blade relative to an airway of a patient during use.

17. The laryngoscope of claim 12, wherein the suction tube is extendable into and retractable out of a field of view of a camera of the laryngoscope.

18. An intubation method, comprising:
manually positioning a camera of a hyperangulated laryngoscope to visualize an airway of a patient; and
cleaning a lens of the laryngoscope while the laryngoscope is positioned to visualize the airway of the patient.

19. The method of claim 18, further comprising the step of extending a suction tube from a blade of the laryngoscope to remove debris and/or excess liquid from the airway.

20. The method of claim 18, wherein the step of cleaning and the step of extending are performed using the same hand used in the step of manually positioning.
